(19) United States
(12) Patent Application Publication
(10) Pub. No.: US 2026/0306510 A1
(43) Pub. Date: Oct. 8, 2026

(54) PROCESS FOR PREPARING DISPENSABLE TESTOSTERONE CREAM

(71) Applicant: LAWLEY PHARMACEUTICALS PTY LTD., australia (AU)

(72) Inventor: Michael Buckley, West Leederville, perth, Western (AU)

(21) Appl. No.: 19/482,413

(22) PCT Filed: May 17, 2024
(86) PCT No.: PCT/IB2024/054806

§ 371 Date: Nov. 7, 2025

(30) Foreign Application Priority Data
May 19, 2023 (GB) 2307501.3

(51) Int. Cl. A61K 31/568; A61K 9/00; A61K 9/107; A61K 47/10; A61K 47/12; A61K 47/14; A61K 47/18; A61K 47/22; A61K 47/32; A61K 47/44

(52) U.S. Cl. CPC A61K 31/568; A61K 9/0014; A61K 9/107; A61K 47/10; A61K 47/12; A61K 47/14; A61K 47/18; A61K 47/22; A61K 47/32; A61K 47/44

(57) ABSTRACT

This invention relates to a process of preparing an oil-in-water emulsion comprising testosterone for use in a pump-action dispensing device. It further relates to the oil-in-water emulsion produced by the process of the invention, and the combination of the emulsion with a pump-action dispensing device.

This Invention relates to a process of preparing an oil-In-water emulsion comprising testosterone for use in a pump-action dispensing device. It further relates to the oil-in-water emulsion produced by the process of the invention, and the combination of the emulsion with a pump-action dispensing device.

Testosterone Is the primary androgenic hormone, produced by the ovaries of women and the testes of men, and by the adrenal glands of both sexes. Testosterone plays an important role in sexual arousal, sexual response, libido, maintenance of bone, energy levels and well-being in men and women.

The use of topical testosterone to treat Hypoactive Sexual Desire Disorder (HSDD), also known as Hypoactive Sexual Desire Dysfunction, in postmenopausal women is well established. HSDD has historically been treated using testosterone products which are formulated for use in the treatment of male hypogonadism. A number of these products are packaged in the form of pump-action dispensing devices and are licensed to deliver male-appropriate doses that are applied to the skin of the upper body in adult males.

Testogel®/Androgel® Is a 16.2 mg/g alcohol-based testosterone gel containing ethanol, Carbomer 980, isopropyl myristate, sodium hydroxide and water, supplied in a pump-action dispenser that delivers 1.25 g of gel containing 20.25 mg of testosterone per pump actuation.

Testavan® is a 20 mg/g alcohol-based testosterone gel containing ethanol, propylene glycol, diethylene glycol monoethyl ether, Carbomer 980, trolamine, disodium edetate and water, supplied in a pump-action dispenser that delivers 1.15 g of gel containing 23 mg of testosterone per pump actuation.

Tostran® is 20 mg/g alcohol-based testosterone gel containing ethanol, propylene glycol, isopropyl alcohol, oleic acid, Carbomer 1382, trolamine, butylhydroxytoluene, hydrochloric acid and water, supplied in a pump-action dispenser that delvers 0.5 g of gel containing 10 mg of testosterone per pump actuation.

The minimum dosage per pump actuation provided by these devices are all at least twice the recommended starting dose of 5 mg per day for the treatment of HSDD in postmenopausal women. Overdosage of testosterone is associated with unwanted (“virilising”) side effects in women including acne, hirsutism, deepening of the voice, clitoromegaly (irreversible growth of the clitoris) and androgenic alopecia.

Furthermore, testosterone use in women is a highly individualised therapy and careful dose monitoring and adjustment is required to ensure that blood testosterone levels do not exceed the premenopausal physiological range. The appropriate dose of testosterone for some women may therefore be less than the currently recommended starting done.

All current transdermal alcohol-based gel formulations of testosterone include a penetration enhancer, which increases testosterone absorbance and thus the chance of overdose in women. Additionally, alcohol gels are sticky upon application, have an odour when drying and leave a residual film on the skin, and as such are not cosmetically acceptable for many male and female patients. More recently a testosterone cream for men has been licensed in Australia for application to the scrotum.

AndroForte® is an oil-in-water based testosterone cream, supplied in a collapsible tube. The formulation is delivered using a syringe-based measurement system to enable accurate dosing. The product is indicated for the treatment of testosterone deficiency in male patients and is supplied in a 50 mg/mL testosterone concentration.

Scrotal skin is up to eight-times more receptive to the absorption of testosterone compared to application to the upper body, allowing a lower dosage to be applied. Alcohol-based testosterone gels cannot be applied to the scrotal area of male patients without the risk of burs and irritation to the sensitive skin, and so are applied to the upper body which requires higher dosage sizes and significantly increases the risk of contamination via passive transfer to third-parties, including women and children.

Globally, only a single product has been specifically developed and licensed for the treatment of HSDD in postmenopausal women.

AndroFerne® is a 10 mg/mL oil-in-water based testosterone cream, suppled in a collapsible tube and delivered using a syringe-based measurement system. A 10 mg daily dose of the AndroFerne® has been shown to be effective at treating HSDD in postmenopausal women (R. Goldstat at al., Menopause 2003, 10(5), 390-398, and G. El-Hage at al., Climacteric 2007, 10(4), 335-343). A 5 mg daily dose was shown to restore total serum testosterone (TT) and free testosterone (fT) levels to levels above and within the reference range, respectively, for premenopausal women (E. Fooladi at al., Menopause 2015, 22(1), 44-49).

Tube-based products require manual dose delivery, a dispensing method that is inconvenient for patients. Operation of the syringe requires a significant degree of patient dexterity and a visual inspection to ensure a correct dosa measurement. This leads to a lack of patient compliance, which can result in under- or over-dosing, potentially leading to lack of efficacy or unwanted side-effects. Furthermore, the accuracy of the delivery method makes it unsuitable for delivering doses smaller than 0.5 mL (5 mg), which may be required for some patients.

Therefore, it exists a need in the art to provide a delivery device for dispensing an oil-in-waler emulsion of testosterone in a way that is convenient for patients and ensures precise dosage measurement at the micro-dose scale.

This need would be met by the use of a pump-action delivery device configured to dispense a predefined unit dosage of the emulsion of testosterone with each pump actuation.

When providing a semi-solid pharmaceutical formulation for use in a multi-dose delivery device there are key regulatory requirements that must be adhered to, so as to ensure that a uniform dosage is consistently provided. Meeting the regulatory requirement is highly dependent on the rheological properties of the formulation, and these properties are in turn dependent on the composition of the formulation including the choice of basis and additional excipients.

However, when providing a pharmaceutical formulation the choice of excipients is also key to determining the pharmacological properties related to the active ingredient, such as stability, skin permeability and bioavailability. It is therefore non-trivial to reformulate a semi-solid composition for use in a new delivery device without requiring extensive testing to ensure that the beneficial pharmacological properties are maintained.

As such, there further exists a need in the art for a method of preparing an oil-in-water emulsion comprising testosterone suitable for use with a pump-action dispensing device.

Accordingly, the present invention provides a process for producing an oil-in-water emulsion of testosterone comprising:
(i) providing an oil-in-water emulsion comprising a water phase, an oil phase, an emulsifying agent and testosterone; and
(ii) stirring the emulsion provided in step (i) under vacuum to remove air bubbles to achieve a viscosity of 20,000 to 50,000 mPa·s when measured at 25° C.

The present invention further provides the oil-in-water emulsion of testosterone produced by the process of the invention.

The use of the process of the invention allows for the production of an oil-in-water emulsion that is compositionally equivalent to the AndroFerne®/AndroForte® formulations known in the art, however with a viscosity of between 20,000 to 50,000 mPa·s when measured at 25° C. allowing it to be uniformly dispensed from a pump-action dispensing device. Based on the commonality of components between the compositions it is predicted that the pharmacological and stability profile of the composition will be retained.

Accordingly, the present invention further provides an al-in-water emulsion of testosterone comprising:
10 mg/g or 50 mg/g testosterone;
20 mg/g polyethylene glycol hexadecyl ether;
10 mg/g cetostearyl alcohol;
0.5 mg/g butylated hydroxytoluene;
40 mg/g almond oil;
12 mg/g of a preservative mixture comprising phenoxyethanol and at least one paraben;
1 mg/g citric acid;
12 mg/g triethanolamine;
8 mg/g carbomer
25 mg/g dl-α-tocopheryl acetate; and
water as solvent,
wherein the emulsion has viscosity of 20,000 to 50,000 mPa·s when measured at 25° C.

The oil-in-water emulsions according to the invention are suitable for use in combination with a pump-action dispensing device.

Accordingly, the invention also provides a pump-action dispensing device comprising a container and a dispensing pump, wherein the container is filed with the oil-in-water emulsion of testosterone according to the invention and wherein manual operation of the pumping device dispenses a predefined unit dose of the emulsion.

Providing the oil-in-water emulsion of testosterone according to the invention in a pump-action dispenser, allows for precise and consistent dosing of testosterone to patients.

Accordingly, the present invention further provides the use of a pump-action dispensing device according to the invention for use in a method of treating a condition characterised by reduced testosterone production, in particular female hypoactive sexual desire disorder (HSDD) or male hypogonadism.

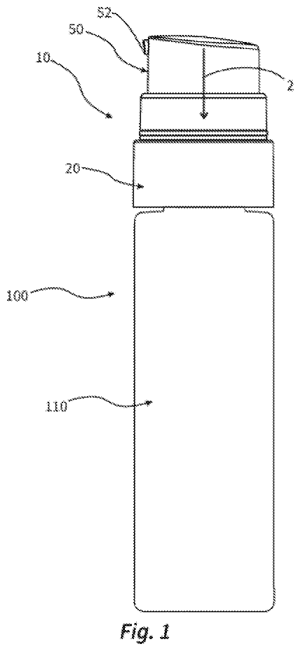

FIG. 1 shows a liquid dispenser 100 for dispensing pharmaceutical liquids, suitable for use in the present invention, reproduced from EP 3 736 049. The liquid dispenser 100 has a liquid reservoir 110 and a discharge head 10, which is fastened to a bott neck or storage connector of the liquid reservoir 110 by means of a thread or some other connection technology.

The discharge head 10 has a base 20 as well as an actuating lever 50 which can be depressed relative to the base 20 in the direction of an actuating direction 2. A discharge opening 52 is provided on this. By depressing the actuating pusher 50, a pump device 12 provided in the discharge head 10 is activated, which pumps liquid from the liquid reservoir 110 to the discharge opening 52.

This Invention relates to a process of preparing an oil-in-water emulsion comprising testosterone for use in a pump-action dispensing device.

Testing of the marketed AndroFerne® composition showed the average viscosity of the product is 149,000 mPa·s when measured at 25° C., in line with the product release specifications for the tube-based delivery system of 65,000 to 230,000 mPa·s. However, a pump-action dispensing device requires a much tighter viscosity range to allow the pump mechanism to operate and achieve the precise dosage requirements required for patient safety.

The Inventors have therefore developed a process for producing an oil-in-water emulsion of testosterone with a viscosity of 20,000 to 50,000 mPa-s when measured at 25° C. Accordingly, this emulsion is suitable for delivery by a pump-action dispensing device, meeting the strict regulatory requirements that are necessary for the delivery of a pharmaceutical product by this method.

The inventors have surprisingly found that increasing the agitation energy imparted into the emulsion significantly reduces the viscosity of the emulsion, bringing it within the range suable for use with a pump-action dispenser (20,000 to 50,000 mPa-s when measured at 25° C.).

A preferred method of measuring the viscosity of the emulsion is using a Brookfield DV2T LV Viscometer, with a thermostatic water both set to 25° C. and a LVT spindle at a speed of 9 rpm, in which an 18-19 g sample of the emulsion is transferred into a glass tube ensuring that no air bubbles are formed during the addition, the sample and the spindle are allowed to equilibrate for at least 10 minutes prior to the initiation of measurement and the viscosity is recorded at 2 minutes.

The stirring time and speed dictate the agitation energy imparted into the emulsion and it is important that these parameters are controlled to provide the target viscosity, whist also ensuring that the overall timescale of the process is industrially applicable.

It is preferred that the emulsion is stirred for at least 2 hours. Preferably, the emulsion is stirred for less than 10 hours. Most preferably the emulsion is stirred for 3 to 4 hours.

It is further preferred that the emulsion is stirred at a speed of 1,200 to 1,500 rpm. During the process stirring may be performed independency at 1,200 or 1,500 rpm. Stirring may be performed intermittently during the process.

The Inventors additionally identified that air bubbles in the oil-In-water emulsion were negatively impacting the dosage precision when the emulsion was dispensed from a pump-action dispensing device. They surprisingly found that application of a vacuum during the process can be used to produce an emulsion which is substantially free from air bubbles.

Preferably, the vacuum is applied at a pressure of −4 to −9 kPa. Preferably, during the formation of the emulsion the pressure is applied at −4 to −7 kPa. Preferably a pressure of −9 kPa Is applied following the formation of the final emulsion.

The order of the steps in the process and the temperature at which they are performed may also have an effect on the rheological properties of the emulsion.

Preferably, the oil-in-water emulsion is prepared in step (i) by combination of the oil phase, the water phase and the emulsifying agent followed by subsequent addition of testosterone. More preferably, the oil phase and the water phase are combined at a temperature of 75 to 80° C., followed by cooing to a temperature of 25 to 30° C. prior to the addition of testosterone.

Preferably the testosterone is added in micronised form.

The invention further relates to the oil-in-water emulsion produced by the process of the invention.

The emulsion of the invention consists of an oil-phase dispersed in a water phase. Preferably the oil used is a vegetable oil, most preferably almond oil.

Preferably the emulsion comprises testolactone in a concentration of 1 to 100 mg/g. Most preferably the concentration of testosterone is 10 mg/g or 50 mg/g.

Preferably, the emulsion has a viscosity of 20,000 to 50,000 mPa s when measured at 25° C. It is more preferred that the viscosity of the emulsion is 35,000 to 45,000 mPa s when measured at 25° C. Most preferably the viscosity of the emulsion is 40,000 mPa s when measured at 25° C.

Preferably, the emulsion is substantially free from air bubbles.

Preferably the pH of the emulsion is in the range 5 to 7. More preferably the pH of the emulsion is 5.5.

Preferably the emulsion further comprises a thickening agent. Preferably the thickening agent is carbomer, most preferably carbomer 980.

Preferably the emulsion further comprises a stabiliser. Preferably the stabiliser comprises at least one fatty alcohol. Most preferably the stabiliser is cetostearyl alcohol.

Preferably the emulsion further comprises an anti-oxidant. Preferably the anti-oxidant is butylated hydroxytoluene.

Preferably, the emulsion further comprises an acidifying agent. Preferably, the acidifying agent is citric acid.

Preferably the emulsion further comprises a neutralising agent preferably the neutralising agent is triethanolamine.

Preferably the emulsion further comprises an emollient. Preferably the emollient is dl-α-tocopheryl acetate (vitamin E acetate).

Preferably the emulsion further comprises a preservative. Preferably the preservative comprises phenoxyethanol and at least one paraben. Preferably, the parabens are selected from methylparaben, ethylparaben, propylparaben, butylparaben and combinations thereof. Most preferably, the preservative comprises phenoxyethanol (70 to 75% w/w), methylparaben (14.5 to 16.5% w/w), ethylparaben (3.7 to 4.3% w/w), propylparaben (1.7 to 2.3% w/w) and butylparaben (1.7 to 2.3% w/w). Examples of suitable preservative mixtures include Phenonip and Kemaben 4 IB.

Preferably, the emulsion comprises a water phase, an oil phase, an emulsifying agent, a thickening agent, a stabiliser, a preservative, an anti-oxidant, an acidifying agent a neutralising agent and testosterone.

In an embodiment, the emulsion consists of a water phase, an oil phase, an emulsifying agent, a thickening agent, a stabiliser, a preservative, an anti-oxidant, an acidifying agent a neutralising agent and testosterone.

The preferred emulsion comprises:
10 mg/g or 50 mg/g testosterone;
20 mg/g polyethylene glycol hexadecyl ether;
10 mg/g cetostearyl alcohol;
0.5 mg/g butylated hydroxytoluene;
40 mg/g almond oil;
12 mg/g of a preservative mixture comprising phenoxyethanol and at least one paraben;
1 mg/g citric acid;
12 mg/g triethanolamine;
8 mg/g carbomer;
25 mg/g dl-α-tocopheryl acetate; and
water as solvent,
wherein the emulsion has viscosity of 20,000 to 50,000 mPa·s when measured at 25° C.

It is further preferred that the emulsion does not comprise any of ethanol, isopropanol or propylene glycol.

The present invention additionally relates to a pump-action device containing an oil-in-water emulsion according to the invention.

The pump-action device according to the invention comprises a container and a dispensing pump, wherein the container is filled with the oil-in-water emulsion according to the invention and wherein manual operation of the pumping device dispenses a predefined unit dose of the emulsion.

Examples of pump action dispensers suitable for dispensing semi-solid pharmaceutical preparations such as the oil-in-water emulsion of testosterone according to the invention, are well known in the art. For example, EP 3 736 049 discloses a liquid dispenser with a discharge head, as shown in FIG. 1.

Airless pump-action dispensing devices allow for consistent metered dosing of small volumes and provide an improved stability profile over traditional pump-action dispensers by reducing the exposure of the contained formulation to air.

The preferred pump-action device is airless.

The size of each individual unit dose of testosterone and total number of doses is controlled by the specifications of the pump-action device.

Preferably the container of the pump-action device is 10 to 100 mL in volume, more preferably 50 mL in volume.

Preferably the pump-action device is configured to dispense a dose of 0.1 to 1 g of the emulsion per pump actuation, more preferably a dose of 0.1 to 0.5 g of the emulsion per pump actuation. A dose of less than 0.5 g is considered to be a “micro-doe”. Most preferably, the device is configured to dispense 0.25 g of the emulsion per pump actuation.

In an embodiment, the pump-action device is configured to dispense a dose of 1 to 10 mg of testosterone per pump actuation, preferably 2.5 mg of testosterone per pump actuation. In another embodiment, the pump-ac ion device is configured to dispense a dose of 5 to 50 mg of testosterone per pump actuation, preferably 12.5 mg of testosterone per pump actuation.

The present invention also relates to the use of the pump-action device for use in a method of treating a condition characterised by reduced testosterone production.

Particular conditions charactered by reduced testosterone production include female hypoactive sexual desire disorder (HSDD) and male hypogonadism, in particular HSDD in postmenopausal women.

Preferably, when used to treat female patients the pump-action device is used to dispense 2.5 mg, 5.0 mg, 7.5 mg or 10 mg of testosterone to the patient in need thereof using a single or multiple pump actuations as required. Preferably, 5 mg is used as a starting dose which is then titrated upwards or downwards based on individual patient's needs. Preferably, the dosage is achieved by dispensing 0.25 g of the emulsion per pump actuation, wherein the concentration of testosterone in the emulsion is 10 mg/g.

Preferably, when used to treat male patients the concentration of testosterone in the oil-in-water emulsion is 50 mg/g.

Preferably, when used to treat male patients the pump-action device is used to dispense 12.5 mg, 25.0 mg, 37.5 mg or 50 mg of teasterone to the patient in need thereof using a single or multiple pump actuations as required. Preferably, 25 mg is used as a starting dose which is then titrated upwards or downwards based on individual patient's needs. Preferably, the dosage is achieved by dispensing 0.25 g of the emulsion per pump actuation, wherein the concentration of testosterone in the emulsion is 50 mg/g. A does of 25 mg or 50 mg can also be achieved by dispensing 0.5 g of the emulsion per pump actuation, wherein the concentration of testosterone in the emulsion is 50 mg/g.

Preferably the oil-in-water emulsion of testosterone is administered topically after being dispensed from the device. In female patients it is preferred that the emulsion is applied to the inner forearms or upper thighs. In male patients it is preferred that the emulsion is applied to the scrotum.

In an embodiment, the emulsion is administered to a male patient by topical application to the torso, wherein the pump action device is used to dispense 100 mg, 150 mg or 200 mg of testosterone to the patient in need thereof using a single or multiple pump actuations as required. Preferably, 150 mg is used as a starting dose which is then titrated upwards or downwards based on individual patient's needs. Preferably, the dosage is achieved by dispensing 0.5 g of the emulsion per pump actuation, wherein the concentration of testosterone in the emulsion is 50 mg/g.

The present invention will now be described with reference to the following examples which are not intended to be limiting.

### EXAMPLES

### Example 1

Preparation of Oil-In-Water Emulsion of Testosterone (10 mg/g)

In a Becomix RW 2000S the water phase was prepared by adding 466.8 kg of purified water to 600 g anhydrous citric acid, followed by heating to 78 to 80° C. and mixing for 2 minutes using pump mode and internal mixing function, speed 1,200 rpm, scrapers 30 rpm (right), −4 kPa vacuum. An amount of 4.8 kg of carbomer 980 was added to the water phase with stirring during mixing, followed by a further 10 to 20 minutes of mixing using pump mode and internal mixing function, speed 1,200 rpm, scrapers 30 rpm (automatic rotation), −4 to −7 kPa vacuum, at 78 to 80° C.

Oil phase prepared using a STERIDOSE 200 by melting 12 kg polyethylene glycol hexadecyl ether, 6 kg cetostearyl alcohol, 300 g butylated hydroxytoluene, 24 kg of almond oil, and 7.2 kg of Kemaben 4 IB, at 78 to 80° C. and mixing for 30 minutes.

Oil phase was added to the water phase with mixing during addition by scrapers 30 rpm (right), with an active vacuum of −4 to −7 kPa, at 75 to 80° C.

Emulsion homogenised for 5 minutes, using homogenising mode and internal mixing function, speed 1,500 rpm, scrapers 30 rpm (right), −4 to −7 kPa vacuum, at 75 to 80° C., followed by cooling to below 30° C., with scrapers 10 rpm (right) and a −4 to −7 kPa vacuum.

An amount of 6 kg of micronised testosterone added with mixing during addition using pump mode and powder suction function (active vacuum starting value −4 kPa), speed 1,200 rpm, scrapers 30 rpm (right), at 25° C. API transfer hoses rinsed with 2×5 kg purified water.

Emulsion mixed for 5 minutes, using pump mode and internal mixing function, speed 1,200 rpm, scrapers 30 rpm (right), −5 kPa vacuum, at 25° C.

An amount of 15 kg of dl-∝-tocopheryl acetate added with mixing during addition using pump mode and internal mixing function, speed 1,200 rpm, scrapers 30 rpm (right), −5 kPa vacuum, at 25° C., followed by a further 2 minutes of mixing.

TEA-water solution prepared by mixing 7.2 kg of triethanolamine (TEA) with 40 kg of purified water. TEA-water solution added to the emulsion with mixing during addition using pump node and internal mixing function, speed 1,200 rpm, scrapers 30 rpm (right), −5 kPa vacuum, at 25° C.

Emulsion mixed for 15 minutes with only scrapers 15 rpm (right), −9 kPa vacuum at 25° C., followed by discharge CIP mode, speed 800 rpm, scrapers 10 rpm (right), +4 kPa overpressure.

The resulting 600 kg emulsion was observed to be a homogenous soft cream without air bubbles, free of visible undissolved particles. It was fled into an 800 L intermediate container under nitrogen atmosphere.

### Example 2

Preparation of Oil-In-Water Emulsion of Testosterone (50 mg/g)

An oil-in-water emulsion of testosterone was produced as in Example 1, however 442.9 kg of purified water was used to prepare the water phase and 30 kg of micronised testosterone was added to the homogenised emulsion.

### Example 3

### Pump Performance Attribute Tests

The emulsions produced in Examples 1 and 2 are filed into Aptar micro+ airless pump systems with a 50 mL container and a 300 μL dispenser adapted to deliver 0.25 mg of the emulsion per pump actuation.

A series of pump performance arbute tests and the respective acceptance criteria are described below.

### Number of Doses Per Dispenser

Device discharged until empty and the number of doses dispensed counted.
Target result: Not less than 200 doses dispensed.

### Uniformity of Delivered Dose, Inter-Container

10 containers taken and a single dose collected from each container. Doses collected at the beginning (from three containers), middle (from four containers) and end (from three containers) of the number of the total number of the target doses (200 doses).

The amount of testosterone in each dose is determined by HPLC.

Target result 10/10 doses within 65-135% of the target testosterone content.

Mean value within 85-115% of the target testosterone content.

### Uniformity of Delivered Dose, Intra-Container

Three doses dispensed from the container and collected separately. Container discharged until 2n+1 doses remain (101 does). Four does dispensed and collected separately. Container discharged until three doses remain. Final three doses dispensed and collected separately.

The amount of testosterone in each dose is determined by HPLC.

Target result 10/10 domes within 65-135% of the target testosterone content
Mean value within 85-115% of the target testosterone content.

### Stability Testing

Content of testosterone, preservative compounds and impurities measured by HPLC initially and after 36 months of storage at less than 25° C.

### Target Result:

TABLE 1

| | Initial | | Shelf-life (36 months) | |
|---|---|---|---|---|
| Testosterone content | 0.95-1.05% | w/v (Example 1) | 0.90-1.10% | w/v (Example 1) |
| | 4.75-5.25% | w/v (Example 2) | 4.50-5.50% | w/v (Example 2) |
| Phenoxyethanol content | 0.797-0.944% | w/v | 0.753-0.987% | w/v |
| Methyl hydroxybenzoate | 0.165-0.207% | w/v | 0.155-0.217% | w/v |
| content | | | | |
| Ethyl hydroxybenzoate content | 0.042-0.054% | w/v | 0.040- 0.056% | w/v |
| Propyl hydroxybenzoate content | 0.019-0.029% | w/v | 0.018-0.030% | w/v |
| Iso-butyl hydroxybenzoate | 0.019-0.029% | w/v | 0.018-0.030% | w/v |
| content | | | | |
| Butyl hydroxybenzoate content | 0.042-0.054% | w/v | 0.040-0.056% | w/v |
| Total hydroxybenzoate content | 0.287-0.373% | w/v | 0.271-0.389% | w/v |

| | | |
|---|---|---|
| Total impurities (sum of | ≤1.0% (Example 1) | ≤1.5% (Example 1) |
| peaks >0.5%) | ≤0.5% (Example 2) | ≤1.0% (Example 2) |

### Tests for Sterility

Measurement of total aerobic microbial count (TAMC), total combined yeasts/mould count (TYMC), presence of Staphylococcus aureus and Pseudomonas aeruginosa.

### Target Result:

TABLE 2

| | |
|---|---|
| Total aerobic microbial count | ≤100 cfu/g |
| (TAMC) | |
| Total combined yeasts/moulds | ≤10 cfu/g |
| count (TYMC)* | |
| Staphylococcus aureus | Absence in 1 g |
| Pseudomonas aeruginosa | Absence in 1 g |

All batches of emulsions produced using the methods defined in Examples 1 and 2 were found to meet the target requirements for number of doses delivered, dose uniformity, initial stability and sterility.

The viscosity of the tested samples was measured using a Brookfleid DV2T LV Viscometer with a thermostatic water bath set to 25° C. and a LVT spindle at a speed of 9 rpm. An 18-19 g sample of the emulsion was transferred into a glass tube ensuring that no air bubbles were formed during the addition. The sample and the spindle were allowed to equilibrate for at least 10 minutes prior to the initiation of measurement. The viscosity was recorded at 2 minutes. The viscosity of al tested samples was found to fall within the target range of 20,000 to 50,000 mPa-s.

### Example 4

### Short-Term Stability Testing

The properties of three batches of emulsion analysed following 6 months in storage at 25° C./60% RH are presented in Table 4. The results of the performance of the pump-action dispensing device used to dispense each batch are presented in Table 5.

The viscosity of each emulsion, number of doses per dispenser, and inter- and intra-container dose uniformity were determined as defined in Example 3. All three batches were found to meet target requirements, with a high degree of consistency observed between batches.

TABLE 4

| | Target | | Batch A | | Batch B | | Batch C | |
|---|---|---|---|---|---|---|---|---|
| pH | 5.0-7.0 | | 6.3 | | 6.3 | | 6.3 | |
| Viscosity | 20,000-50,000 | mPa · s | 26,300 | mPa · s | 28,100 | mPa · s | 30,500 | mPa · s |
| Testosterone content | 0.90-1.10% | w/v | 1.03% | w/v | 1.03% | w/v | 1.04% | w/v |
| Phenoxyethanol content | 0.753-0.987% | w/v | 0.853% | w/v | 0.857% | w/v | 0.867% | w/v |
| Methyl hydroxybenzoate | 0.155-0.217% | w/v | 0.186% | w/v | 0.188% | w/v | 0.189% | w/v |
| content | | | | | | | | |
| Ethyl hydroxybenzoate | 0.040-0.056% | w/v | 0.047% | w/v | 0.047% | w/v | 0.048% | w/v |
| content | | | | | | | | |
| Propyl hydroxybenzoate | 0.018-0.030% | w/v | 0.023% | w/v | 0.023% | w/v | 0.023% | w/v |
| content | | | | | | | | |
| Iso-butyl hydroxybenzoate | 0.018-0.030% | w/v | 0.022% | w/v | 0.023% | w/v | 0.023% | w/v |
| content | | | | | | | | |
| Butyl hydroxybenzoate | 0.040-0.056% | w/v | 0.047% | w/v | 0.047% | w/v | 0.048% | w/v |
| content | | | | | | | | |
| Total hydroxybenzoate | 0.271-0.389% | w/v | 0.325% | w/v | 0.328% | w/v | 0.332% | w/v |
| content | | | | | | | | |

| Total impurities (sum of | ≤1.5% | 0.426% | 0.471% | 0.560% |
|---|---|---|---|---|
| peaks >0.5%) | | | | |

TABLE 5

| | | Batch | Batch | Batch |
|---|---|---|---|---|
| | Target | A | B | C |
| Intra-container | 10/10 doses within | Conforms | Conforms | Conforms |
| uniformity | 65-135% of the | | | |
| | target testosterone | | | |
| | content | | | |
| | Mean value within | Conforms | Conforms | Conforms |
| | 85-115% of the | | | |
| | target testosterone | | | |
| | content | | | |
| Inter-container | 10/10 doses within | Conforms | Conforms | Conforms |
| uniformity | 65-135% of the | | | |
| | target testosterone | | | |
| | content | | | |
| | Mean value within | Conforms | Conforms | Conforms |
| | 85-115% of the | | | |
| | target testosterone | | | |
| | content | | | |
| Doses per | ≥200 doses | 222 | 215 | 221 |
| dispenser | | | | |

1. A process for preparing an oil-in-water emulsion of testosterone, the method comprising:
(i) providing an oil-in-water emulsion comprising a water phase, an oil phase, an emulsifying agent and testosterone; and
(ii) stirring the emulsion provided in step (i) under vacuum to remove air bubbles to achieve a viscosity of 20,000 to 50,000 mPa·s when measured at 25° C.

2. A process for preparing an oil-in-water emulsion of testosterone as claimed in claim 1, wherein the emulsion is stirred for at least 2 hours.

3. A process for preparing an oil-in-water emulsion of testosterone as claimed in claim 1 or 2, wherein the emulsion is stirred at 1,200 to 1,500 rpm.

4. A process for preparing an oil-in-water emulsion of testosterone as claimed in any preceding claim, wherein the vacuum is applied at a pressure of −4 to −9 kPa.

5. A process for preparing an oil-in-water emulsion of testosterone as claimed in any preceding claim, wherein an oil-in-water emulsion is prepared in step (i) by combination of the oil phase, the water phase and the emulsifying agent followed by subsequent addition of testosterone.

6. A process for preparing an oil-in-water emulsion of testosterone as claimed in claim 6, wherein the oil phase and the water phase are combined at a temperature of 75 to 80° C., followed by cooling to a temperature of 25 to 30° C. prior to the addition of testosterone.

7. A process for preparing an oil-in-water emulsion of testosterone as claimed in any preceding claim, wherein the emulsion comprises testosterone in a concentration of 1 to 100 mg/g.

8. A process for preparing an oil-in-water emulsion of testosterone as claimed in any preceding claim, wherein the emulsion further comprises a thickening agent, a stabiliser, a preservative, an anti-oxidant, an acidifying agent and/or a neutralising agent.

9. A process for preparing an oil-in-water emulsion of testosterone as claimed in any preceding claim, wherein the emulsion consists of a water phase, an oil phase, an emulsifying agent, a thickening agent, a stabiliser, a preservative, an anti-oxidant, an acidifying agent, a neutralising agent and testosterone.

10. A process for preparing an oil-in-water emulsion of testosterone as claimed in any preceding claim, wherein the pH of the emulsion is 5 to 7.

11. An oil-in-water emulsion of testosterone, obtainable by the process of any preceding claim.

12. An oil-in-waler emulsion of testosterone comprising:
10 mg/g or 50 mg/g testosterone;
20 mg/g polyethylene glycol hexadecyl ether;
10 mg/g cetostearyl alcohol;
0.5 mg/g butylated hydroxytoluene;
40 mg/g almond oil;
12 mg/g of a preservative mixture comprising phenoxyethanol and at least one paraben;
1 mg/g citric acid;
12 mg/g triethanolamine;
8 mg/g carbomer;
25 mg/g dl-α-tocopheryl acetate; and
water as solvent,
wherein the emulsion has viscosity of 20,000 to 50,000 mPa·s when measured at 25° C.

13. A pump-action dispensing device comprising a container and a dispensing pump, wherein the container is filed with the oil-in-water emulsion of claims 11 or 12 and wherein manual operation of the pumping device dispenses a predefined unit dose of the emulsion.

14. The pump-ac don dispensing device as claimed in claim 13, wherein the pump-action dispensing device is airless.

15. The pump-action dispensing device as claimed in claims 13 or 14, for use in a method of treating a condition characterised by reduced testosterone production, in particular female hypoactive sexual desire disorder (HSDD) or male hypogonadism.
